(19) United States
(12) Patent Application Publication
(10) Pub. No.: US 2024/0050206 A1
(43) Pub. Date: Feb. 15, 2024

(54) COBALT-PLATINUM BASED DENTAL ALLOY MATERIALS

(71) Applicant: Ivoclar Vivadent AG, Schaan (LI)

(72) Inventor: Patrick McCabe, Schaan (LI)

(21) Appl. No.: 18/448,895

(22) Filed: Aug. 11, 2023

Related U.S. Application Data
(60) Provisional application No. 63/371,216, filed on Aug. 11, 2022.

(51) Int. Cl. A61C 13/09; A61C 13/08; C22C 30/00

(52) U.S. Cl. CPC A61C 13/09; A61C 13/081; C22C 30/00

(57) ABSTRACT

Provided are dental alloys comprising platinum, cobalt, and gold and optionally further comprising one or more of ruthenium, gallium, manganese, chromium and silicon, wherein the gold makes up from 0.5 to 5.0 wt % of the total alloy. The alloys have desirable castability and ease of manufacture. The alloys can be used in manufacture of dental articles comprising the alloy and porcelain materials, and used for various dental restorations.

### CROSS REFERENCE TO RELATED APPLICATION

This application claims priority to U.S. Provisional Application No. 63/371,216, filed on Aug. 11, 2022, the disclosure of which is incorporated herein by reference.

### BACKGROUND OF THE DISCLOSURE

Various alloys comprising gold, palladium, platinum, copper and others have been used as dental alloy materials. Alloys are generally used as cast metal substrates with a veneer of porcelain to provide a composite to make various dental articles. It is important to have a strong bond between the alloy and the porcelain. For alloys containing precious metals, cost can be a limiting factor. Many palladium alloys have been used, but while palladium alloys provide some desirable properties, such as thermal expansion properties, its cost and other limitations highlight a need for improved dental alloy compositions.

### SUMMARY OF THE DISCLOSURE

The present disclosure provides a noble metal-based dental alloy composition. In an aspect, this disclosure provides a cobalt and platinum-based alloy.

Exemplary alloys of the present disclosure comprise 40 wt % to 50 wt % Co, 17 wt % to 20 wt % Pt, and 0.5 wt % to 5 wt % Au, and optionally, one or more of Ru, Cr, Mn, Si, and Ga.

The alloy is free of copper and silver, and palladium. Copper could produce a dark oxide, silver could discolor the ceramic, palladium would increase cost.

In many embodiments, the alloy compositions are non-magnetic and exhibit desirable thermal expansion properties, and have improved castability.

In an aspect, this disclosure provides alloys which have desirable thermal properties to be compatible with bonding to ceramics, including glass ceramics.

In an aspect, this disclosure provides an alloy which has gold in the range of 0.5 to 5.0 wt % and has CTE in the range that is compatible with bonding to ceramics, including glass ceramics, e.g., lithium silicate ceramics, and optionally, has improved castability.

In an aspect, this disclosure provides a porcelain fused to metal dental alloy that is easy to cast, metal finished, and exhibits a light oxide capable of bonding to dental porcelains, e.g., commercially available dental porcelains. The dental alloys can be used in many types of dental restorations.

The present dental alloy can be manufactured by routine melt process, cast into a bar and/or rolled to the required thickness or alternatively, by the atomization and compression method of U.S. Pat. No. 5,799,386 to Ingersoll et al. entitled Process Of Making Metal Castings, issued Sep. 1, 1998, the disclosure of which is incorporated herein by reference.

### BRIEF DESCRIPTION OF THE FIGURES

For a fuller understanding of the nature and objects of the disclosure, reference should be made to the following detailed description taken in conjunction with the accompanying figures.

FIG. 1 is a representation of the tensile properties of the present alloy compositions.

FIG. 2 is a representation of the coefficient of linear expansion for alloy compositions.

FIG. 3 is a representation of the melting range values for some of the present alloy compositions.

FIG. 4 is a representation of Vickers microhardness values for some the present alloy compositions.

FIG. 5 is a side-by-side comparison of an oxide according to the present disclosure and an exemplary known oxide.

### DETAILED DESCRIPTION OF THE DISCLOSURE

Although claimed subject matter is described herein in terms of certain embodiments and examples, other embodiments and examples, including embodiments and examples that do not provide all of the benefits and features set forth herein, are also within the scope of this disclosure. Various structural, logical, and process step may be made without departing from the scope of the disclosure.

Every numerical range given throughout this specification includes its upper and lower values, as well as every narrower numerical range that falls within it, as if such narrower numerical ranges were all expressly written herein, and, unless described otherwise, every value is included to the tenth of the value of the lower limit.

The present disclosure provides cobalt-based platinum containing alloy compositions. The alloy compositions have one or more of the following desirable properties: absence of ferromagnetism, good thermal expansion, ease of manufacture, and improved castability.

In many embodiments, the alloy composition comprises 40 wt % to 50 wt % Co, 17 wt % to 20 wt % Pt, and 0.5 wt % to 5 wt % Au, where the wt % is relative to the total weight of the alloy. The alloy composition may additionally comprise one or more of Ru, Cr, Mn, Si, and Ga.

In some embodiments, the alloy compositions comprise 40 wt % to 50 wt % cobalt, 17 wt % to 25 wt % platinum, and 0.5 wt % to 5 wt % gold, 5 wt % to 6.5 wt % ruthenium, 28 wt % to 30 wt % chromium, 0.5 wt % to 1.0 wt % silicon, 0.6 wt % to 1.0 wt % manganese and 0.2 wt % to 0.4 wt % gallium, optionally to make up to a 100%.

In many embodiments, the alloy compositions comprise cobalt, platinum and gold, wherein platinum and gold together make up from 18 to 26 wt % of the alloy and gold is not more than 5% wt % of the alloy, and the alloy may further optionally comprise ruthenium, chromium, silicon, manganese and gallium.

In many embodiments, the alloy composition comprises from 0.5 to 5% wt gold and all ranges and values therebetween, where the wt % is relative to the total weight of the alloy. In embodiments, the alloy compositions may comprise 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt % gold, and may further comprise 40 wt % to 50 wt % cobalt, 17 wt % to 25 wt % platinum, and may further optionally comprise 5 wt % to 6.5 wt % ruthenium, 28 wt % to 30 wt % chromium, 0.5 wt % to 1.0 wt % silicon, 0.6 wt % to 1.0 wt % manganese, and 0.2 wt % to 0.4 wt % gallium, and optionally the total weight percent is 100%.

In some embodiments, the alloy composition comprises 40 wt % to 45 wt % cobalt, 18.5 wt % to 19.5 wt % platinum, and 0.5 wt % to 1.5 wt % gold, 5.5 wt % to 6.0 wt % ruthenium, 28 wt % to 30 wt % chromium, 0.6 wt % to 0.7 wt % silicon, 0.2 wt % to 0.4 wt % gallium, and 0.7 wt % to 0.9 wt % manganese, and optionally the total weight percent is 100%, where the wt % is relative to the total weight of the alloy.

An exemplary alloy composition (Alloy 37) of the present disclosure is provided in Table 1.

TABLE 1

| ELEMENT | WEIGHT % |
|---|---|
| Platinum | 18.9 |
| Ruthenium | 5.7 |
| Cobalt | 43.5 |
| Chromium | 29.0 |
| Manganese | 0.8 |
| Silicon | 0.7 |
| Gallium | 0.3 |
| Gold | 1.1 |

Some properties of the exemplary alloy composition (Alloy 37) are provided in Table 2.

TABLE 2

| PROPERTIES | RESULTS | |
|---|---|---|
| Melting Range | 1405-1435° | C. |
| CTE | | |
| 25-500° C. | 14.1 | |
| 25-600° C. | 14.4 | |
| Vickers Hardness | | |
| Porcelain Cycled | 295 | |
| Tensile Properties | | |
| 0.2% Offset YS | 385 | M Pa |
| Modulus | 228 | M Pa |
| Elongation | 8% | |
| Density | 9.27 | g/cc³ |

The present compositions are non-magnetic and can be used for dental applications.

Alloys according to various embodiments of the present disclosure exhibit one or more of the following properties.
Non-Magnetic
Can be classified as a noble alloy according to the Identalloy program containing at least 25% of noble metals including platinum, ruthenium and gold.
Meet ISO 22674: Dentistry—Metallic materials for fixed and removable restorations and appliances as a Type 4 alloy.

The alloys exhibit a thermal expansion of 13.8 10⁻⁶ K⁻¹ to 15.1×10⁻⁶ K⁻¹.

Further, the alloy compositions of the present disclosure exhibit desirable physical properties including Vickers Hardness of 295 to 395 HV, Tensile strength of 500 to 1000 MPa, and Elongation of 2 to 12%.

Exemplary thermal properties include CTE at 500° C. of 13.9 to 14.3×10⁻⁶ K⁻¹ CTE at 600° C. of 14.1 to 14.5×10⁻⁶ K⁻¹, and Melting range of 1390 to 1450° C.

The present alloy exhibits non-magnetic properties and good corrosion resistance.

In an aspect, this disclosure provides a dental article comprising the dental alloys described herein. For example the dental article may comprise a dental alloy comprising 40 wt % to 50 wt % cobalt, 17 wt % to 20 wt % platinum, and 0.5 wt % to 5 wt % gold, and optionally further comprising one or more of ruthenium, chromium, manganese, silicon, and gallium, where the wt % is relative to the total weight of the alloy. In various embodiments, the dental alloy in the dental article may comprise 40 wt % to 50 wt % cobalt, 17 wt % to 25 wt % platinum, 0.5 wt % to 5 wt % gold, 5 wt % to 6.5 wt % ruthenium, 28 wt % to 30 wt % chromium, 0.5 wt % to 1.0 wt % silicon, 0.6 to 1.0 wt % manganese and 0.2 to 0.4 wt % gallium, and optionally the total weight percent is 100%, where the wt % is relative to the total weight of the alloy. In other various embodiments, the dental article may comprise a dental alloy comprising 40 wt % to 45 wt % cobalt, 18.5 wt % to 19.5 wt % platinum, 0.5 wt % to 1.5 wt % gold, 5.5 wt % to 6.0 wt % ruthenium, 28 wt % to 30 wt % chromium, 0.6 wt % to 0.7 wt % silicon, 0.2 to 0.4 wt % gallium, and 0.7 to 0.9 wt % manganese, and optionally the total weight percent is 100%, where the wt % is relative to the total weight of the alloy; or a dental alloy comprising 43.5 wt % cobalt, 18.9 wt % platinum, 1.1 wt % gold, 5.7 wt % ruthenium, 29.0 wt % chromium, 0.7 wt % silicon, 0.3 wt % gallium, and 0.8 wt % manganese, and optionally the total weight percent is 100%, where the wt % is relative to the total weight of the alloy.

The dental alloy article may comprise a dental alloy as described herein and a ceramic or glass ceramic bonded onto the dental alloy substrate. The dental article may be fabricated as a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector. The dental alloy may be cast into a single 2 unit, 3-unit or 4-unit or longer span bridges.

The dental alloy of the present disclosure can be cast by routine dental processes and may be recast by routine dental laboratory procedures. The alloying elements form an oxide on the cast metal surface. This dark oxide layer, can affect the apparent color of the ceramic or glass ceramic veneering layer. The alloy system of the present invention may include elements added to regulate the amount and color of the oxide layer and melting characteristics, selected from the group including, but not limited to: silicon, manganese, and/or gallium.

In an embodiment, a wax framework is designed and fabricated in a slightly reduced anatomic size, reduced in size proportionally based on the shape and size of the dental item and taking the planned veneer into consideration. This reduced size of the wax framework results in an alloy framework that will accommodate the ceramic layer that will be layered onto the alloy, which results in a restoration that will be proportional to the dentition that the restoration will replace. Sprues and a reservoir can be attached to the framework, and a phosphate-bonded investment material is poured around the pattern to create a mold. The wax is then burned out in a suitable oven, leaving the mold and a shaped cavity. The alloy is then cast into the cavity left from the wax pattern and allowed to cool. When the alloy has cooled in the mold, the casting is divested and the framework is removed. The surface can be finished using dental burs or stones. The surface is blasted with aluminum oxide and cleaned with distilled water. The framework is oxidized in a ceramic oven at 900° C. with a 1 minute hold without vacuum. The resulting oxide produces an acceptable bond but in an embodiment, the oxide can be blasted off without affecting the bond to the ceramic.

The steps of the method described in the various embodiments and examples disclosed herein are sufficient to carry out the methods of the present disclosure. Thus, in an example, a method consists essentially of a combination of the steps of the methods disclosed herein. In another example, a method consists of such steps.

The following Statements provide various non-limiting embodiments of the present disclosure:
Statement 1. A dental alloy comprising 40 wt % to 50 wt % cobalt, 17 wt % to 25 wt % platinum, and 0.5 wt % to 5 wt % gold, and optionally further comprising one or more of ruthenium, chromium, manganese, silicon, and gallium, wherein the wt % is relative to the total weight of the alloy.
Statement 2. A dental alloy of Statement 1 comprising 40 wt % to 50 wt % cobalt, 17 wt % to 20 wt % platinum, 0.5 wt % to 5 wt % gold, 5 wt % to 6.5 wt % ruthenium, 28 wt % to 30 wt % chromium, 0.5 wt % to 1.0 wt % silicon, 0.6 to 1.0 wt % manganese, and 0.2 to 0.4 wt % gallium, wherein the total weight percent is 100%.
Statement 3. A dental alloy of Statement 2 comprising 40 wt % to 45 wt % cobalt, 18.5 wt % to 19.5 wt % platinum, 0.5 wt % to 1.5 wt % gold, 5.5 wt % to 6.0 wt % ruthenium, 28 wt % to 30 wt % chromium, 0.6 wt % to 0.7 wt % silicon, 0.2 to 0.4 wt % gallium, and 0.7 to 0.9 wt % manganese, wherein the total weight percent is 100%.
Statement 4. A dental alloy of Statement 1 comprising 43.5 wt % cobalt, 18.9 wt % platinum, 1.1 wt % gold, 5.7 wt % ruthenium, 29.0 wt % chromium, 0.7 wt % silicon, 0.3 wt % gallium, and 0.8 wt % manganese, wherein the total weight percent is 100%.
Statement 5. A dental alloy of Statement 1, wherein the total weight percent of platinum and gold combined is 19 to 20 wt % of the total alloy.
Statement 6. A dental alloy of any of the preceding Statements, wherein the CTE is 13.9 to 14.3.
Statement 7. A dental article comprising the dental alloy of any one of the preceding Statements and a ceramic or glass ceramic bonded onto the dental alloy substrate.
Statement 8. A dental article of Statement 7, fabricated as a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector.
Statement 9. A dental article of Statement 7 or Statement 8, wherein the ceramic or glass ceramic comprises lithium silicate.
Statement 10. A process for manufacture of a dental article comprising a dental alloy and a ceramic or a glass ceramic comprising: casting the dental alloy according to one of claims 1-6 to form a body; applying the ceramic or glass ceramic to the body; and firing the body and applied ceramic or glass ceramic to form the dental article.
Statement 11. A process of Statement 10, wherein the dental article is a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector.
Statement 12. A process of claim 10, wherein the ceramic or glass ceramic comprises lithium silicate.
Statement 13. A dental alloy, comprising 40 wt % to 50 wt % cobalt, 17 wt % to 25 wt % platinum, and 0.5 wt % to 5 wt % gold; and the dental alloy optionally further comprises one or more of: ruthenium, chromium, manganese, silicon, and gallium, wherein the wt % is relative to the total weight of the dental alloy.
Statement 14. A dental alloy of Statement 13, wherein the weight percent of platinum is 17 wt % to 20 wt %, and the dental alloy further comprises: 5 wt % to 6.5 wt % ruthenium, 28 wt % to 30 wt % chromium, 0.5 wt % to 1.0 wt % silicon, 0.6 to 1.0 wt % manganese, and 0.2 to 0.4 wt % gallium, wherein the total sum of the weight percent of the dental alloy is 100%.
Statement 15. The dental alloy of claim 2, wherein the weight percent of cobalt is 40 wt % to 45 wt %, the weight percent of platinum is 18.5 wt % to 19.5 wt %, the weight percent of gold is 0.5 wt % to 1.5 wt %, the weight percent of ruthenium is 5.5 wt % to 6.0 wt %, the weight percent of chromium is 28 wt % to 30 wt %, the weight percent of silicon is 0.6 wt % to 0.7 wt %, the weight percent of gallium is 0.2 to 0.4 wt % gallium, and the weight percent of manganese is 0.7 to 0.9 wt %.
Statement 15. A dental alloy of Statements 13 or 14, wherein the weight percent of cobalt is 43.5 wt %, the weight percent of platinum is 18.9 wt %, the weight percent of gold is 1.1 wt %, the weight percent of ruthenium is 5.7 wt %, the weight percent of chromium is 29.0 wt %, the weight percent of silicon is 0.7 wt %, the weight percent of gallium is 0.3 wt %, and the weight percent of manganese is 0.8 wt %.
Statement 16. A dental alloy of any one of Statements 13 to 15, wherein the total weight percent of platinum and gold combined is 19 to 20 wt % of the total alloy.
Statement 17. A dental alloy of any one of Statements 13 to 16, wherein the CTE is 13.9 to 14.3.
Statement 18. A dental article comprising the dental alloy of any one of Statements 13 to 17 and a ceramic or glass ceramic bonded onto the dental alloy substrate.
Statement 19. A dental article of Statement 18, wherein the dental article is fabricated as a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector.
Statement 20. A dental article of Statements 18 or 19, wherein the ceramic or glass ceramic comprises lithium silicate.
Statement 21. A process for manufacture of a dental article comprising a dental alloy and a ceramic or a glass ceramic comprising: casting the dental alloy according to any one of Statements 13 to 17 to form a body; applying the ceramic or glass ceramic to the body; and firing the body and applied ceramic or glass ceramic to form the dental article.
Statement 22. A process of Statement 21, wherein the dental article is a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector.
Statement 23. A process of Statements 21 or 22, wherein the ceramic or glass ceramic comprises lithium silicate.

The following example is presented to illustrate the present disclosure. It is not intended to be limiting in any matter.

### EXAMPLE

This example provides a description of preparation and properties of some alloys of the present disclosure.

The alloy was melted by induction after reaching a temperature of 1600° C. and with a hold time of 30 minutes. The melt was poured into a sand mold to produce alloy rods. The rods were cut into ingots.

Testing was conducted according to ISO 22674 and ISO 9693 standards. Tests included determination of mechanical (tensile) properties, thermal expansion coefficient, Vickers microhardness, melting range (solidus, liquidus), and debonding/crack initiation test.

The debonding/crack initiation test was conducted according to ISO 9693. Six metallic specimens with a dimension of (25±1) mm×3±0.1) mm×(0.5±0.05) mm were cast from the alloy and prepared according to the manufacturer's instructions. Ceramic was applied with a thickness of (1.1±0.1) thickness and a length of (8±0.1) mm. Specimens were fired in a ceramic oven. The specimens were placed in a testing jig with the ceramic facing down. A force was applied and recorded at failure. The fracture force was inserted into a formula and a final strength was determined. The strength must be greater than 25 MPa to satisfy the ISO standard.

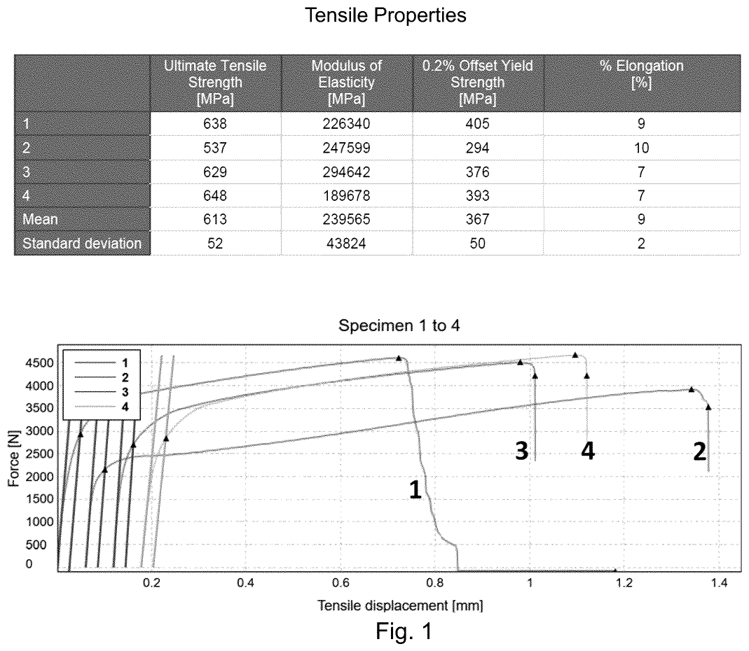

Results are shown in FIGS. 1-5.

FIG. 1 is a representation of the tensile properties determined by the testing. The results listed in the chart represent the mechanical properties of the alloy. The Ultimate Tensile Strength is the strength of the alloy at fracture. The Modulus of Elasticity is stiffness of the alloy and ability to resist deflection. The 0.2% Offset Yield Strength is the strength at which the alloy permanently deforms and will not return to the original length. The Percent Elongation is the amount of elongation of the alloy after fracture.

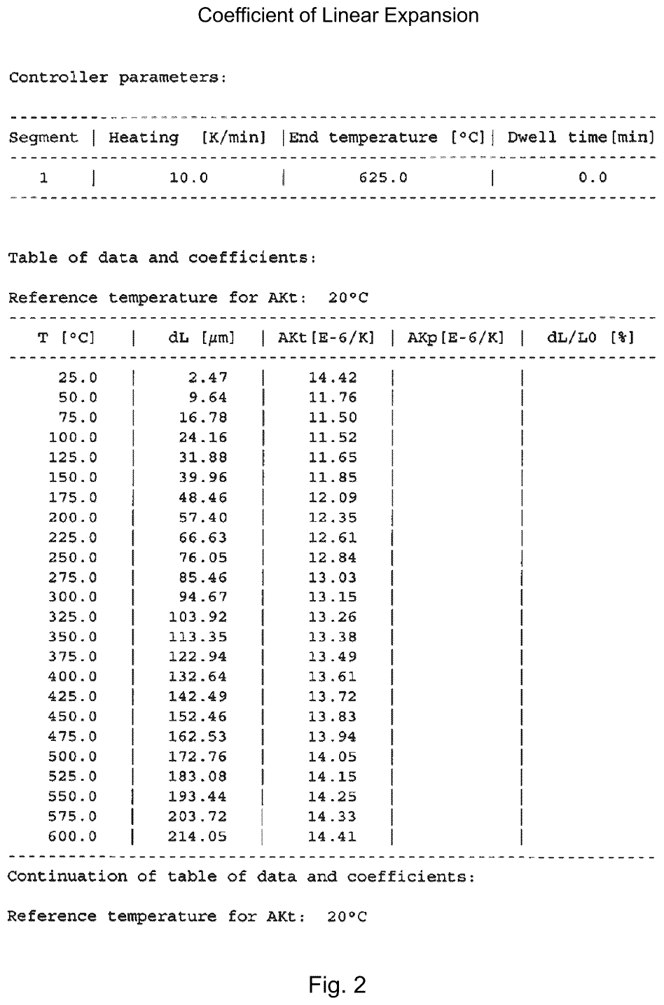

FIG. 2 is a representation of test results for the coefficient of thermal expansion. The results listed represent the amount of expansion and contraction of the alloy during the ceramic firing process.

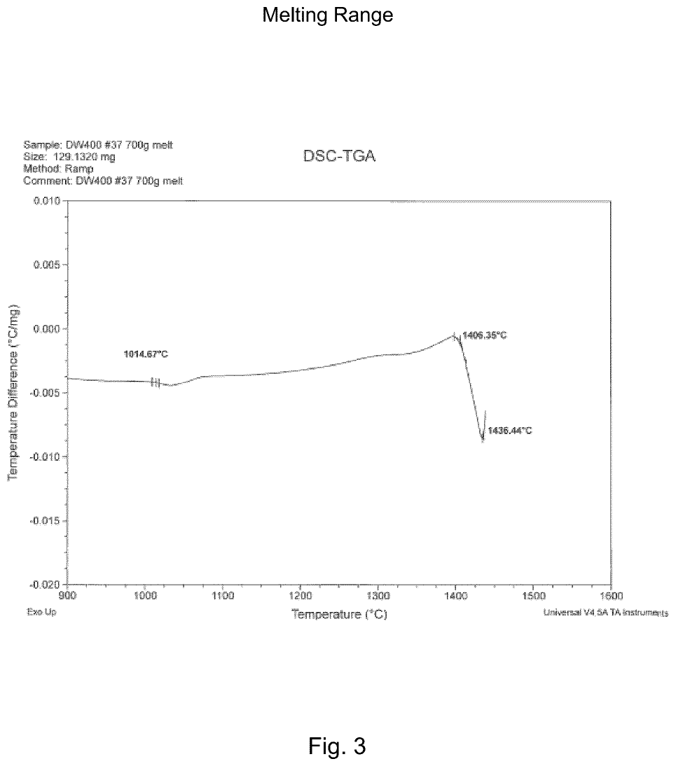

FIG. 3 shows the melting range. The results listed represent the range over which the alloy melts. It starts melting at the solidus temperature and is completely melted at the liquidus temperature.

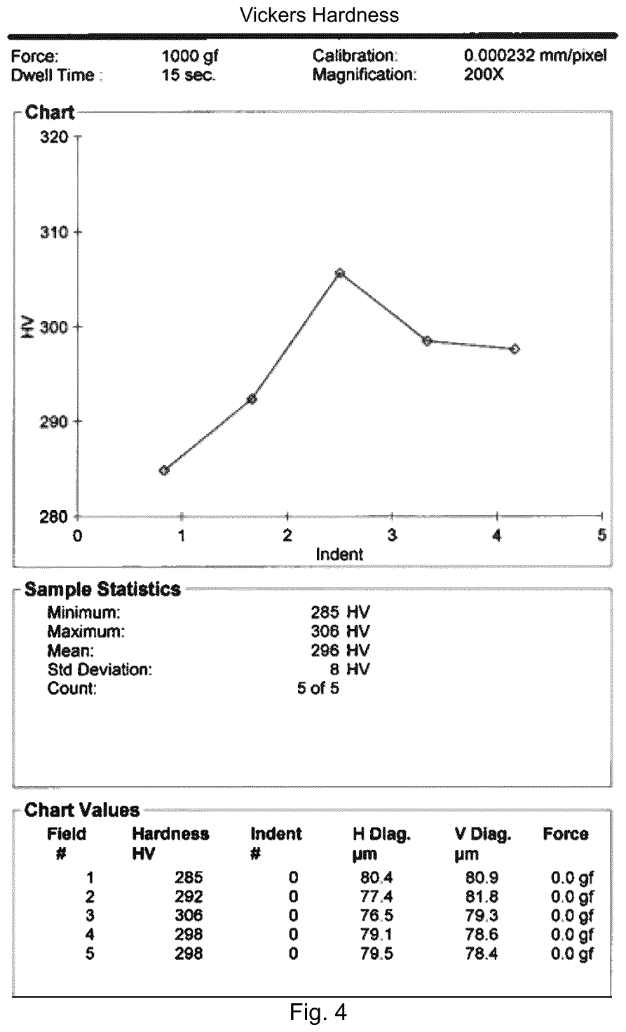

FIG. 4 shows the Vickers microhardness of the alloy determined using a diamond indenter.

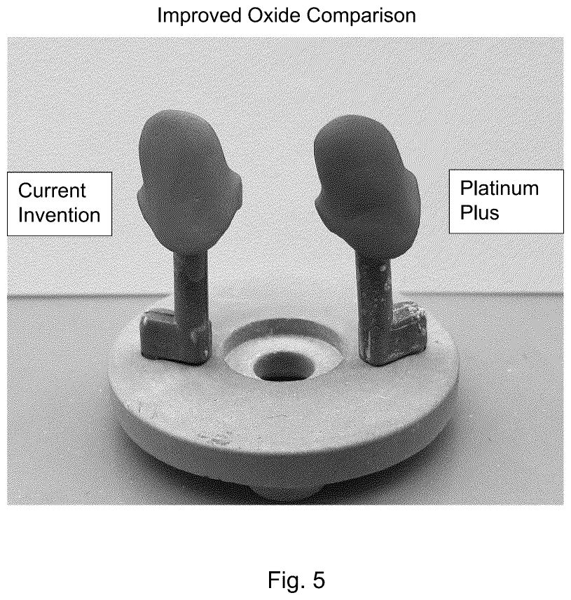

FIG. 5 shows a visual comparison between an oxide prepared according to the present disclosure and an oxide known in the art. The oxides are shown after being fired in a ceramic oven. The oxide prepared according to the present disclosure is lighter in color. A lighter oxide is easier to cover the oxide color with dental opaque.

Although the present disclosure has been described with respect to one or more particular embodiments and/or examples, it will be understood that other embodiments and/or examples of the present disclosure may be made without departing from the scope of the present disclosure.

1. A dental alloy,
comprising:
40 wt % to 50 wt % cobalt,
17 wt % to 25 wt % platinum, and
0.5 wt % to 5 wt % gold; and
the dental alloy optionally further comprises one or more of:
ruthenium, chromium, manganese, silicon, and gallium,
wherein the wt % is relative to the total weight of the dental alloy.

2. The dental alloy of claim 1,
wherein
the weight percent of platinum is 17 wt % to 20 wt %,
and the dental alloy further comprises:
5 wt % to 6.5 wt % ruthenium,
28 wt % to 30 wt % chromium,
0.5 wt % to 1.0 wt % silicon,
0.6 wt % to 1.0 wt % manganese, and
0.2 wt % to 0.4 wt % gallium,
wherein the total weight percent is 100%.

3. The dental alloy of claim 2, wherein
the weight percent of cobalt is 40 wt % to 45 wt %,
the weight percent of platinum is 18.5 wt % to 19.5 wt %,
the weight percent of gold is 0.5 wt % to 1.5 wt %,
the weight percent of ruthenium is 5.5 wt % to 6.0 wt %,
the weight percent of chromium is 28 wt % to 30 wt %,
the weight percent of silicon is 0.6 wt % to 0.7 wt %,
the weight percent of gallium is 0.2 wt % to 0.4 wt % gallium, and
the weight percent of manganese is 0.7 wt % to 0.9 wt %.

4. The dental alloy of claim 2, wherein
the weight percent of cobalt is 43.5 wt %,
the weight percent of platinum is 18.9 wt %,
the weight percent of gold is 1.1 wt %,
the weight percent of ruthenium is 5.7 wt %,
the weight percent of chromium is 29.0 wt %,
the weight percent of silicon is 0.7 wt %,
the weight percent of gallium is 0.3 wt %, and
the weight percent of manganese is 0.8 wt %.

5. The dental alloy of claim 1, wherein the total weight percent of platinum and gold combined is 19 to 20 wt % of the total alloy.

6. The dental alloy of claim 1, wherein the CTE is 13.9 to 14.3.

7. A dental article comprising the dental alloy of claim 1 and a ceramic or glass ceramic bonded onto the dental alloy substrate.

8. The dental article of claim 7, wherein the dental article is fabricated as a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector.

9. The dental article of claim 7, wherein the ceramic or glass ceramic comprises lithium silicate.

10. A process for manufacture of a dental article comprising a dental alloy and a ceramic or a glass ceramic comprising:
casting the dental alloy according to claim 1 to form a body;
applying the ceramic or glass ceramic to the body; and
firing the body and applied ceramic or glass ceramic to form the dental article.

11. The process of claim 10, wherein the dental article is a crown, bridge, veneer, inlay, onlay, partial crown, fixed partial denture, implant abutment, implant, orthodontic appliance, space maintainer, tooth replacement appliance, splint, dentures, post, teeth, jacket, facing, veneer, facet, cylinder, or connector.

12. The process of claim 10, wherein the ceramic or glass ceramic comprises lithium silicate.
